(19) United States
(12) Patent Application Publication
(10) Pub. No.: US 2026/0069793 A1
(43) Pub. Date: Mar. 12, 2026

(54) ADD-ON DEVICE FOR AN INJECTION DEVICE

(71) Applicant: Sanofi, Paris (FR)

(72) Inventors: Stefan Alt, Frankfurt am Main (DE); Michael Helmer, Frankfurt am Main (DE)

(21) Appl. No.: 19/107,528

(22) PCT Filed: Aug. 28, 2023
(86) PCT No.: PCT/EP2023/073463

§ 371 Date: Feb. 27, 2025

(30) Foreign Application Priority Data
Aug. 30, 2022 (EP) 22315197.8

(51) Int. Cl. A61M 5/315

(52) U.S. Cl. CPC A61M 5/31568; A61M 2205/18; A61M 2205/52

(57) ABSTRACT

Disclosed is an add-on device attachable to an injection device and including a device body fastenable to a portion of the injection device, a sensor operable to quantitatively determine at least one of a position and a movement of a movable component of a drive mechanism of the injection device and operable to generate a respective sensor signal, at least one of a reader and a transceiver being operable to read or to obtain a machine-readable identification of the injection device containing at least one of a device information and a container information, and an electronic module comprising a module processor operable to determine or to calculate a size of the dose of the medicament set or dispensed by the injection device on the basis of the sensor signal and at least one of the device information and the container information.

### CROSS-REFERENCE TO RELATED APPLICATIONS

The present application is the national stage entry of International Patent Application No. PCT/EP2023/073463, filed on Aug. 28, 2023, and claims priority to European Application No. EP 22315197.8, filed on Aug. 30, 2022, the disclosures of which are incorporated herein by reference.

### TECHNICAL FIELD

The present disclosure relates to the field of add-on devices for injection devices, in particular to add on devices attachable to a pen-type injector. In another aspect the disclosure relates to an injection device and to an injection system as well as to a method of monitoring operation of an injection device or injection system.

### BACKGROUND

Drug delivery devices for setting and dispensing a single or multiple doses of a liquid medicament are as such well-known in the art. Generally, such devices have substantially a similar purpose as that of an ordinary syringe.

Drug delivery devices, such as pen-type injectors, have to meet a number of user-specific requirements. For instance, with patients suffering chronic diseases, such as diabetes, the patient may be physically infirm and may also have impaired vision. Suitable drug delivery devices especially intended for home medication therefore need to be robust in construction and should be easy to use. Furthermore, manipulation and general handling of the device and its components should be intelligible and easy understandable. Such injection devices should provide setting and subsequent dispensing of a dose of a medicament of equal or variable size. Moreover, a dose setting as well as a dose dispensing procedure must be easy to operate and has to be unambiguous.

A patient suffering from a particular disease may require a certain amount of a medicament to either be injected via a pen-type injection syringe.

Some drug delivery or injection devices provide selecting of a dose of a medicament of variable size and injecting a dose previously set. Other injection devices provide setting and dispensing of a fixed dose. Here, the amount of medicament that should be injected in accordance to a given prescription schedule is always the same and does not change or cannot be changed over time.

Some injection devices are implemented as reusable injection devices offering a user to replace a medicament container, such as a cartridge. Other injection devices are implemented as a disposable injection device. With disposable injection devices it is intended to discard the entirety of the injection device when the content, i.e. the medicament, has been used up.

In order to control and to supervise administering of medication conducted by users or patients themselves it is beneficial to assist the user by making use of an external electronic device, such as a mobile electronic device, e.g. implemented as a smartphone, a tablet computer, or a smart watch. A software application provided on such external electronic devices may interact with the user and may provide instructions or recommendations to the user of how to correctly use the injection device.

In order to control and to supervise administering of medication conducted by users or patients themselves it is desirable to provide an automated detecting and logging of a repeated and regular use of the drug delivery device. A rather automated recording of doses injected by a user would offer a significant advantage over a manual dose logging in terms of security and convenience.

There exist numerous add-on devices configured for use with injection devices that offer an electronic detection and monitoring of repeated dose injection procedures.

Typically, such add-on devices or auxiliary devices can be detachably connected to the injection device. An add-on device typically comprises a sensor or sensor arrangement operable to detect a date and/or time when the user sets or injects a dose of the medicament. Some add-on devices also provide a quantitative measurement of a size of a dose currently set or dispensed. Some add-on devices are intended for use with a series of injection devices. This may particularly apply with disposable injection devices, that are intended to become discarded after use or after the medicament located therein has been used up.

Generally, there exists a variety of different injection devices, which may be equipped with different drugs or medicaments but are generally operable to be used with one and the same type of an add-on device. While it is common practice to characterize or to label injection devices and/or medicaments of different types and/or of different concentration by a clear and distinct label or identification the detachable connection with an add-on device may impose a disadvantage, in particular when a label or a characteristic portion of the injection device is covered by the add-on device when attached to the injection device.

Some add-on devices may be generally suitable for use with a variety of injection devices. Such injection devices may distinguish by different calibrations of a drive mechanism. Such injection devices may be also equipped with different medicaments, medicament concentrations or medicament amount. In order to have a correct monitoring of use of the injection device it is therefore mandatory to suitably deploy and/or to correctly calibrate the add-on device in the course of attaching the add-on device to the injection device.

It is therefore desirable to provide improvements form add-on devices, injection devices and injection systems, which facilitate a correct, easy and straightforward calibration of the add-on device as well as a correct and failure safe monitoring of repeated uses of the injection device.

### SUMMARY

In one aspect the present disclosure relates to an add-on device for attaching to an injection device. The injection device typically comprises a housing and a drive mechanism for injecting of a dose of a medicament, wherein the medicament is provided in a medicament container, such as a cartridge. The drive mechanism comprises at least one movable component, which is subject to a movement relative to the housing or relative to the medicament container during at least one of setting of the dose and injecting of the dose of the medicament.

The add-on device comprises a device body fastenable to a portion of the injection device. The add-on device further comprises a sensor operable to quantitatively determine at least one of a position and a movement of the movable component relative to at least one of the housing, the medicament container and the device body. The sensor is further operable to generate a respective sensors signal being indicative of the position and/or of the movement of the movable component relative to at least one of the housing, the medicament container and the device body.

The add-on device further comprises at least one of a reader and a transceiver. The reader or transceiver is or are operable to read or to obtain the machine-readable identification of the injection device.

The add-on device further comprises an electronic module comprising a module processor coupled to the sensor and coupled to at least one of the reader and the transceiver. The module processor is operable to determine or to calculate a size of the dose of the medicament, which size is set or dispensed by the injection device. Determining or calculating a size of the dose is based on the sensor signal(s) and on at least one of the device information and the container information contained in the machine-readable identification.

Specifically and by reading or obtaining the machine-readable identification of the injection device the add-on device can be autonomously provided with at least one of the device information and the container information provided by or stored in the machine-readable identification. This way, the electronic module and hence the add-on device may automatically obtain at least one of the device information and the container information on the basis of which determining or calculation of the dose size has to be conducted.

With some examples the container information is indicative of at least one of a type of a medicament contained in the medicament container a concentration of a pharmaceutical substance of the medicament contained in the container, a manufacturing date of the medicament, a use by date of the medicament or, a LOT number of the medicament or medicament container and/or environmental parameters, such as temperature, humidity or radiation intensity to which the medicament container has been exposed.

The container information may further include information about the type of the container in which the medicament is currently stored or contained. Insofar, the container information may contain container specific information, such as a type of a primary container, e.g. filled with the medicament or a type of a secondary container, e.g. a container part of an injection device, such as a cartridge holder configured to receive a primary container and configured for attachment to a body of a housing of an injection device.

The container information is contained in the machine-readable identification. The machine-readable identification may be provided in one of a number of different forms or formats. With some examples the machine-readable identification is provided in printed form. It may comprise visual or optically readable information, such as letters, signs or codes, e.g. in form of a one-dimensional barcode or 2-dimensional data matrix code. Here, the reader or transceiver comprises an optical reader, such as an imaging system capable to capture the visual or optically readable information of the machine-readable identification.

With other examples the machine-readable identification is provided in an electric or electronic storage. It may be provided in a digital storage document such as a memory of an integrated circuit. Accordingly, the reader or transceiver on the electronic module comprises an electronic or digital reader operable to read the digital storage of the machine-readable identification. With some examples the machine-readable identification comprises one of an active or passive RFID, NFC or UWB tag readable by a respective RFID, NFC or UWB reader or transceiver of the electronic module.

With further examples the machine-readable identification comprises a magnetic code, readable by a respective magnetic reading or transceiving device of the electronic module.

With some examples the machine-readable identification is printed on a label, which may be adhered or integrated on or in one of the medicament container and the housing of the injection device.

The device information may comprise information or data with regard to the type of the injection device. The device information may be specific about a type of an injection mechanism or drive mechanism of the injection device. The device information may thus characterize the dose setting and/or dose dispensing operability of the respective injection device. The device identification may further contain data or information about suitable medicaments or medicament containers to be exclusively used with the respective injection device.

With some examples the drive mechanism comprises a piston rod, which in order to expel a predefined amount of a medicament has to move a predefined longitudinal displacement relative to the housing of the injection device. The drive mechanism is characterized by a particular feeding rate of its piston rod per unit of the medicament dose. The feeding rate of the drive mechanism has to match with the medicament and/or with a medicament container in use with the drive mechanism or injection mechanism.

By way of the reader or transceiver the add-on device is capable to obtain or to read the device information and/or the container information as provided on or inside the injection device. Typically, reading of the device information and/or the container information is enabled and becomes exclusively possible when the add-on device is attached to the injection device. Insofar and upon installing or mounting the add-on device to or on the injection device the reader or transceiver of the add-on device can be autonomously triggered to read or to obtain the machine-readable identification thereby obtaining at least one of the device information and the container information.

The device information and/or the container information can then be used to calibrate the dose size determination or dose size calculation to be conducted by the module processor of the electronic module of the add-on device.

The sensor of the add-on device is typically capable to provide at least one or a series of sensor signals being indicative of the position or movement of the movable component of the drive mechanism relative to at least one of the housing, the medicament container and the device body. The movable component may allow for a unique and/or unequivocal determination of a size of a dose currently set or dispensed or injected by the drive mechanism of the injection device. The movable component may be provided with an encoding, which is movable relative to the sensors such that the sensor is capable to count or to detect a movement of the movable component in the course of setting of a dose and/or injecting or dispensing of a dose of the medicament.

The measurable position and/or the measurable movement of the movable component of the drive mechanism is unequivocally assigned with a respective size of a dose. The assignment between the degree or magnitude of movement and the size of a dose may differ between different drive mechanisms and may differ with different medicament containers.

For instance, with a medicament container of a standard size a longitudinal feeding movement of the piston rod of the drive mechanism leads to a dispensing of a standard size of a dose of the medicament. With a medicament container comprising a smaller or larger diameter the same longitudinal feeding movement of the piston rod may lead to a dispensing or injecting of a reduced amount or larger amount of the medicament from the medicament container.

Therefore, it is important to have precise knowledge of the type of the medicament container and of the type of the drive mechanism when deriving or calculating a size of a dose of the medicament on the basis of the sensor signals provided by the sensor of the add-on device.

With the present add-on device, and by the above described acquisition of at least one of a device information and a container information a somewhat automated calibration of the dose size determination or dose size calculation can be provided. Hence, the add-on device itself is capable to derive and/or to process medicament container specific information and/or device specific information, e.g. in the course of assembly to the injection device. In response to obtain the container information and/or the device information the add-on device may be configured to conduct a calibration routine. This way the measurable sensor signals as obtained by the sensor of the add-on device can be translated or re-calculated into correct dose size information.

According to a further example the at least one of the reader and the transceiver of the add-on device comprises a wireless near field transceiver operable to wirelessly communicate with the machine-readable identification of the injection device in order to obtain at least one of the device information and the container information. Typically, the reader and/or the transceiver is operable to obtain the device information and to obtain the container information. The device information and the container information may be contained or provided in one and the same machine-readable identification. With some examples the device information and the container information are provided in or by separate machine-readable identifications provided on or inside the injection device.

When the transceiver of the add-on device is implemented as a near field transceiver the machine-readable identification is typically provided as a near field transceiver or near field communication tag as well. The machine-readable identification may comprise an electronic identifier such as a RFID tag, a NFC tag or UWB tag that enables near field communication with a RFID reader, a NFC reader and/or UWB reader. Here, the transceiver of the add-on device comprises or constitutes a respective RFID reader, near field communication (NFC) or ultra wide band (UWB) reader.

Use of a wireless near field transceiver is beneficial in that reading of the machine-readable identification will be only possible when the transceiver and hence the add-on device is within a predefined geometric or spatial range of the machine-readable identification. This way, a configuration or calibration of the add-on device may only take place when the distance between the machine-readable identification and the reader or transceiver is smaller than the near field communication range of the wireless near field transceiver.

According to a further example the at least one of the reader and the transceiver comprises a wireless local range transceiver. The wireless local range transceiver is operable to wirelessly communicate with an external electronic device to receive the machine-readable identification of the injection device from or via the external electronic device. The local range transceiver may comprise a transmission range that is larger than the transmission range of the near field transceiver. The local range transceiver may comprise a radiofrequency transceiver. It may comprise a Bluetooth transceiver or a Bluetooth low energy (BLE) transceiver. It may also comprise a Wi-Fi transceiver or the like wireless transceiver allowing for a local range signal transmission with the external electronic device.

Typically, the external electronic device comprises one of a smart phone, a smartwatch and a tablet computer. With a communication link between the add-on device and the external electronic device it is also possible that the device information and/or container information as provided by the machine-readable identification is provided to the electronic module of the add-on device via the external electronic device. Here, the external electronic device may be equipped with a reader or transceiver operable to read-out the machine-readable identification of the injection device. In response to such a read-out of the machine-readable identification by the external electronic device the external electronic device may then be capable to extract the device information and/or the container information from the machine-readable identification and to transmit at least one or both of the device information and the container information to the add-on device via the local range transceiver of the add-on device. This way, the add-on device is indirectly provided with the information or data contained in or provided by the machine-readable identification of the injection device. With this example the add-on device may be void of a near field transceiver and does not necessarily have to be capable to obtain or to read the machine-readable identification by itself. Here, the external electronic device may operate as a relay, which reads or obtains the device information and/or the container information from the machine-readable identification and which is further operable to provide or to transmit the gathered information to the add-on device.

Of course, the local range transmission between the electronic module of the add-on device and the external electronic device requires some kind of an authentication procedure or pairing, such that unauthorized reading or transmission of device information and/or container information can be effectively controlled and/or prevented.

According to a further example the module processor of the add-on device is operable to convert the sensor signal into dose size information by taking into account at least one of the device information and the container information.

Typically, at least one of the device information and the container information is used to calibrate the conversion of the sensor signal conducted by the module processor.

In effect and with the capability to automatically obtain the device information and/or the container information the add-on device can be generally used with a variety of different injection devices that distinguish by at least one of their drive mechanism and/or by their medicament container. Conversion of the sensor signal into the dose size then automatically takes into account the device specific information and/or the container specific information. In view of this it is no longer necessary to deploy the add-on device or the electronic module thereof manually when a user intends to use the add-on device with a specific type of an injection device or with a specific type of a medicament.

According to a further example the module processor is operable to convert a first sensor signal into a first dose size information when provided with at least one of a first device information and a first container information. The module processor is operable to convert the first sensor signal into a second dose size information when provided with at least one of a second device information and a second container information. Here, the second device information or the second container information distinguishes from the first device information and the first container information, respectively. Accordingly, the second dose size information distinguishes from the first dose size information. To conclude, with one and the same sensor signal there will be derived different dose size information depending on the device information or container information as obtained from the machine-readable identification of the injection device. Accordingly, the device information and/or the container information provided by the machine-readable identification of the injection device can be appropriately used to calibrate the calculation or determination of the dose size to be derived from the sensor signal of the sensor of the add-on device.

According to a further example the electronic module further comprises a module memory connected to the module processor. The module memory and/or the module processor is or are operable to store at least one of the device information and the container information as stored information and/or to store a dosing history, wherein the dosing history includes a number of dose sizes dispensed or injected by the injection device and determined or calculated by the module processor.

With some examples the electronic module is operable to read or to obtain at least one of the device information and the container information at least once or only once, namely when mounting the add-on device to the injection device. In the course of mounting or attaching the add-on device to the injection device the electronic module is configured to read or to obtain the relevant device information and/or container information and to store the respective information in the module memory of the electronic module. This way, the device information and/or container information obtained from the machine-readable identification in the course of assembly of the add-on device to the injection device can be at least temporally stored in the module memory. This way and as long as the add-on device rests or remains on the injection device a repeated reading or obtaining of device information and/or container information is not necessary.

The stored information in the module memory can be used to calculate or to determine the size of the dose on the basis of the sensor signal generated by the sensor and processed by the module processor in the course of setting of the dose and/or dispensing of the dose of the medicament.

Furthermore, also the dosing history, hence a series of dose sizes, e.g. sequentially dispense or injected by the injection device can be stored in the module memory of the electronic module. Typically, and by way of a communication link to an external electronic device the recorded data, in particular the dosing history, hence the date and time as well as the amount of medicament set, dispensed and/or injected by the injection device can be synchronized with and/or transmitted to the external electronic device.

This way, the information gathered or derived by the add-on device and being indicative of the repeated use of the injection device can be easily shared with and can be further processed by a physician and/or a healthcare provider.

According to a further example the stored information as stored in the module memory includes at least one of a stored device information and a stored container information. With some examples the module memory is capable to store the device information as well as the container information as obtained from the machine-readable identification.

According to a further example the module processor is operable to determine or to calculate a residual amount of the medicament contained in the medicament container. This calculation or determination is typically based on the dosing history and on at least one of the device information and the container information.

On the basis of the dosing history the electronic module and/or the module processor is or are operable to determine a total amount of medicament dispensed or injected from a medicament container during repeated or successive dose injection procedure as conducted with the injection device. On the basis of at least one of the device information and the container information as obtained from the machine-readable identification of the injection device the module processor and/or the electronic module also obtains relevant information of the initial amount of medicament provided initially in the medicament container. By comparing, e.g. by subtracting the amount of medicament dispensed or injected during the recorded dosing history from the initial amount of medicament provided in the medicament container the residual amount of medicament left inside the container can be electronically derived or calculated.

According to a further example the module processor and/or the electronic module may be operable to repeatedly read or to obtain the machine-readable identification of the injection device. For instance, each detection of a dose setting action or dose dispensing action as detectable by the sensor of the add-on device may trigger a respective readout of the machine-readable identification with the help of at least one of the reader and the transceiver of the add-on device. This way the module processor is also operable to compare at least one of the device information and the container information actually or repeatedly obtained from the machine-readable identification with the stored information as stored in the module memory, e.g. upon an initializing procedure of the add-on device conducted before, during or after mounting of the add-on device to the injection device.

Comparing of the momentary container information as repeatedly obtained from the machine-readable identification with previously stored device information and/or previously stored container information provides a security check if the add-on device is still attached to one and the same injection device.

In case that a comparison between container information or device information actually obtained from the machine-readable identification does not match with previously stored information the electronic module may conduct a predefined action in order to prevent recording of an incorrect dose size.

According to a further example the module processor is operable to activate a signal generator of the add-on device and/or to generate or to trigger an alert signal if at least one of the device information actually obtained from the machine-readable identification does not match with the stored device information and/or if the container information actually obtained from the machine-readable identification does not match with the stored container information. If this check as conducted by the module processor should fail the module processor may be operable to activate the signal generator of the add-on device and/or to trigger generation of an alert signal.

Activation of the signal generator may induce generation of at least one of an audible, a visual and/or a palpable signal. Accordingly, the signal generator may be configured to produce one of a noise or sound, an optical signal, such as a flashlight or blinking light and a vibration signal. With further examples an alert signal may be triggered or generated by or through signal transmission with the external electronic device. Here, and e.g. by way of the wireless local range transceiver, the add-on device may induce a respective alert or signal generation through wireless alert signal transmission with the external electronic device. This way, the add-on device may be void of an own signal generator but may effectively use a signal generator of the external electronic device, which is triggered through wireless alert signal transmission from the add-on device to the external electronic device.

In any case the add-on device may be configured to attract a user's attention in case that one of the device information and the container information actually obtained or read from the machine-readable identification does not or no longer match with respective device information or container information previously stored in the module memory or storage.

According to a further example the module processor is operable to discard or to interrupt determination or calculation of the residual amount of the medicament contained in the medicament container if at least one of the device information obtained from the machine-readable identification does not match with the stored device information and/or if at least one of the container information obtained from the machine-readable identification does not match with the stored container information. Here, discarding or interruption of the calculation of the residual amount of the medicament may be conducted instead or concurrently with the activation of a signal generator or triggering of an alert signal in case that at least one of actually obtained device information or container information does no longer match with predefined or previously stored device information or container information.

According to a further example the module processor is operable to acquire and/or to request at least one of the device information and the container information before, during or after processing the sensor signals. Recording or receiving an initial sensor signal after a certain time interval, during which the sensor of the add-on device has been inactive, may automatically trigger a repeated readout of the machine-readable identification thus revealing at least one of the device information and the container information.

Acquisition of the device information or container information and/or readout of the machine-readable identification may be also triggered during or immediately after processing of the sensor signal(s).

With some examples the injection device, in particular the drive mechanism of the injection device, comprises a locking mechanism by way of which operation of the drive mechanism can be interlocked or blocked. The locking mechanism may be implemented mechanically or electromechanically. The locking mechanism may be operable or controlled by the add-on device. Here, the add-on device may comprise a locking controller, which is either electrically or mechanically coupled to the locking mechanism of the drive mechanism when the add-on device is correctly attached to the injection device. The locking controller may be operated by the electronic module in order to activate and/or to deactivate the locking mechanism.

With some examples and when the module processor of the electronic module of the add-on device should determine that at least one of the device information and the container information as provided by the machine-readable identification does no longer match with predefined and/or previously stored device information or container information the module processor may be operable to activate the locking mechanism of the drive mechanism thereby preventing any further use of the injection device. In this way, a user is effectively hindered to set or to dispense an incorrect dose of the medicament.

According to a further example the module processor is operable to generate use-related data being indicative of a status of use of the injection device. The module processor is further operable to transmit the use-related data to the machine-readable identification and/or to store the use-related data in a memory of an electronic identifier of the machine-readable identification. With some examples the machine-readable identification comprises at least one of a RFID tag, NFC tag or UWB tag with a writable memory. This way, use-related data being indicative of a status of use of the injection device can be locally stored in the memory of the electronic identifier thereby allowing to store use-related data of the injection device and/or of the medicament container directly on or in the injection device.

For instance, the use-related data may be indicative of the total amount of medicament extracted or dispensed by the injection device during use with the add-on device attached to the injection device. Use-related data may be further indicative of a dosing history of the injection device. It may contain data such as a point of time or data at which as dose dispensing action took place. It may further contain data about the amount of medicament dispensed with each dosing or injection action. By storing the use-related data directly in the machine-readable identification it may become allowable to detach the add-on device from the injection device even before the entirety of the medicament as provided in the medicament container has been dispensed or injected from the medicament container.

Later on, and when the add-on device should be reattached to the injection device the reader or transceiver of the add-on device may be capable to read or to obtain not only the device information and the container information but also the use-related data from the machine-readable identification. Then, the module processor may be configured to resume the recording of the dosing history as well as the calculation or determination of the residual amount of medicament left in the medicament container.

According to a further aspect the present disclosure also relates to an injection device for setting of a dose of a medicament. The injection device comprises a housing and a drive mechanism for injecting of the dose of the medicament provided in a medicament container. The drive mechanism comprises at least one movable component, which is subject to a movement relative to the housing or medicament container during at least one of setting of the dose and injecting of the dose of the medicament. The injection device further comprises a machine-readable identification comprising an electronic circuit including a writable memory containing at least one of a device information and a container information. Typically, the writable memory is readable and/or writable by at least one of a reader and a transceiver of an electronic module of an add-on device as described above. Hence, the writable memory of the machine-readable identification may be written, read and/or overwritten by the add-on device when the add-on device is attached to the injection device. This way, not only original device information or container information but also use-related data, e.g. being indicative of the prior use of the injection device or medicament container, e.g. containing a dosing history can be stored in principle in the writable memory. When the injection device is reattached or reconnected with an add-on device respective information can be retrieved by the device, thus allowing to resume the recording of a dosing history and/or to resume calculation of a residual amount of medicament left in a medicament container.

According to a further example the device information stored in the machine-readable identification includes at least one tolerance parameter being indicative of a device specific geometric or positional tolerance of the at least one movable component measured or determined during manufacturing or assembly of the injection device. Such device specific geometric or positional tolerances of the at least one movable component may have an influence of the precision of the measurement conducted by the sensor of the add-on device. Since geometrical and/or positional tolerances of the at least one movable component may vary from injection device to injection device and may distinguish with a number of injection devices or drive mechanisms it is of particular advantage to precisely measure or to determine the device specific geometrical and/or positional tolerances of the at least one movable component with each injection device or drive mechanism and to store such device specific geometrical and/or positional tolerances in the machine-readable identification.

This way, any device specific geometrical positional tolerances of the movable component can be taken into account when calculating or deriving a size of the dose on the basis of the sensor signal as obtained from the sensor that measures or determines at least one of a position and a movement of the movable component relative to at least one of the housing, the medicament container and the device body.

With some examples the movable component is one of a dose dial being rotatable relative to the housing for setting of a dose, a dial sleeve rotatable relative to the housing during setting of the those or dispensing of the dose, a drive sleeve or a drive member, which is subject to a rotational and/or longitudinal motion relative to the housing during at least one of setting of the dose and dispensing of the dose. With some examples the movable component is a clicker or clicking element operable to generate an audible sound recordable by the sensor of the add-on device. With further examples the movable component is a piston rod operably engaged with a piston of the medicament container for displacing the piston relative to the medicament container during injecting of the dose. With further examples the movable component is a single dose indicating member, whose movement and/or position relative to the housing or device body is indicative of the size of a dose currently set. With further examples the movable component is a last dose member, whose position relative to the housing of the injection device is directly indicative of the residual amount of medicament left in the medicament container or cartridge.

With another example the present disclosure further relates to an injection system. The injection system comprises a first injection device, wherein the first injection device comprises a housing and a first drive mechanism for injecting of a dose of a medicament provided in a first medicament container. The first drive mechanism comprises at least one movable component, which is subject to a movement relative to the housing or relative to the first medicament container during at least one of setting of the dose and injecting of the dose of the medicament.

The first injection device also comprises a machine-readable identification containing at least one of a first device information and a first container information. The injection system further comprises an add-on device, e.g. as described above. The add-on device comprises a device body fastenable to a portion of the injection device. The add-on device further comprises a sensor operable to quantitatively determine at least one of a position and a movement of the movable component relative to at least one of the housing, the first medicament container and the device body, typically when the device body is attached and/or fixed to the injection device. The sensor is operable to generate a respective sensor signal(s) in response to the detection of a position and/or of a movement or velocity of movement of the movable component of the injection device.

The add-on device further comprises at least one of a reader and a transceiver being operable to read or to obtain the machine-readable identification of the injection device and further comprises an electronic module comprising a module processor coupled to the sensor and coupled to at least one of the reader and the transceiver. The module processor is operable to determine or to calculate a first size of the dose of the medicament, which is set or dispensed by the first injection device. Calculation or determination of the first size of the dose of the medicament is conducted on the basis of the sensor signal(s) and on the basis of at least one of the first device information and the first container information contained in the machine-readable identification.

In effect, the injection system comprises an add-on device as described above connected or coupled to an injection device as described above.

With a further example the injection system comprises a second injection device. The second injection device may be of the same or similar operability than the first injection device. The second injection device may distinguish from the first injection device e.g. by the medicament, the medicament container and/or by the transmission or translation of the drive mechanism, e.g. the transmission between a dose setting or dose dispensing component relative to the feeding motion of e.g. a piston rod of the drive mechanism.

The second injection device comprises a housing and a second drive mechanism for injecting of a dose of a medicament provided in a second medicament container. The second drive mechanism comprises at least one movable component, which is subject to a movement relative to the housing or relative to the second medicament container during at least one of setting of the dose and injecting of the dose of the medicament. The second injection device further comprises a second machine-readable identification containing at least one of a second device information and a second container information. Here, the module processor of the add-on device is operable to determine or to calculate a second size of the dose of the medicament, which is set or dispensed by the second injection device, on the basis of the sensor signal and on the basis of at least one of the second device information and the second container information contained in the second machine-readable identification.

Here, the add-on device is automatically adaptable to the second injection device. The adaptation of the add-on device may be conducted autonomously, e.g. by reading or otherwise obtaining at least one of the second device information and the second container information. The sensor signal(s) as obtained from the sensor of the add-on device are then translated or calibrated on the basis of the second and hence modified device information or container information.

According to a further example of the injection device or of the injection system the machine-readable identification comprises an electronic identifier. The electronic identifier comprises an electronic circuit. The electronic circuit comprises an antenna for wireless communication with at least one of the reader and the transceiver of the add-on device. The electronic circuit further comprises a memory to store at least one of the device information and the container information. The machine-readable identification may comprise at least one of a passive or active RFID tag, NFC tag or UWB tag.

Typically and according to a further example the machine-readable identification comprises a processor connected to an antenna and connected to a memory in order to store information in the memory and/or to read information from the memory.

According to a further example the memory of the machine-readable identification is a writable memory and is further configured to store use-related data being indicative of a state of use of the injection device. The use-related data may contain the dosing history, the total amount of medicament dispensed from the medicament container and the like data being indicative of a use of the injection device.

According to a further example the present disclosure relates to a method of monitoring operation of an injection device configured for injecting of a dose of a medicament. The method comprises the steps of reading or obtaining at least one of a device information and a container information from a machine-readable identification of an injection device by at least one of a reader and a transceiver of an add-on device configured for attaching to the injection device. The method further comprises the step of quantitatively determining at least one of the position and a movement of a movable component of the drive mechanism of the injection device, wherein the drive mechanism is operable to inject a dose of the medicament provided in a medicament container. Determining at least one of the position and a movement of the movable component is typically conducted by a sensor of the add-on device.

In response to the quantitative detection, measuring or determination of the position and/or of the movement of the movable component the sensor generates a respective sensor signal being indicative of the position and/or of the movement of the movable component. The method further comprises the step of determining or calculating a size of the dose of the medicament, which size has been set or dispensed by the injection device. Determining or calculating the size is conducted on the basis of the sensor signal and on the basis of at least one of the device information and the container information as obtained from the machine-readable identification.

Typically, the method of monitoring operation of the injection device is to be conducted by an injection device, by an add-on device and/or by an injection system as described above. Insofar, all features, effects and benefits as described above in connection with any one of the add-on device, the injection device and the injection system equally apply to the method of monitoring operation of the injection device; and vice versa.

According to a further aspect the present disclosure also relates to a computer program, e.g. a computer program product comprising computer readable instructions, which when executed by a module processor of an add-on device as described above cause the module processor to conduct the steps of the method as described above.

Typically, the computer program is executable by an add-on device as described above. It is further operable to conduct a method of configuring the add-on device as described above. Insofar, the computer program and its computer readable instructions are operable to cause a processor of the add-on device to conduct a method of configuring the add-on device as described above. The computer readable instructions may be executable by a processor of an add-on device as described above.

Additionally, or alternatively and when an external electronic device of an injection system as described above is configured to read-out the machine-readable identification of the injection device the computer program and its computer readable instructions may be at least in part executable by a processor of the external electronic device.

The computer program is to be executed by a processor of an add-on device as described above in order to execute a method of configuring the add-on device as described above and/or in order to monitor and/or record operation of the injection device when the add-on device is attached thereto. Insofar, all features, effects and benefits as described above in connection with the add-on device and/or described in connection with the method of configuring the add-on device as well as any effects, features and benefits as described above in connection with the injection system equally apply to the computer program; and vice versa.

The present disclosure further discloses and proposes a computer program including computer-executable instructions for performing the method according to the disclosed method/device/system in one or more of the examples enclosed herein when the program is executed on a processor, computer or computer network. Specifically, the computer program may be stored on a computer-readable data carrier. Thus, specifically, one, more than one or even all of the method steps as indicated above may be performed by using a computer or a computer network, typically by using a computer program.

The present disclosure further discloses and proposes a computer program product having program code means, in order to perform the method according to the disclosed method/system in one or more of the embodiments enclosed herein when the program is executed on a computer or computer network. Specifically, the program code means may be stored on a computer-readable data carrier.

Further, the present disclosure discloses and proposes a data carrier having a data structure stored thereon, which, after loading into a processor, computer or computer network, such as into a working memory or main memory of the processor, computer or computer network, may execute the method according to one or more of the examples disclosed herein.

The present disclosure further proposes and discloses a computer program product with program code means stored on a machine-readable carrier, in order to perform the method or parts thereof according to one or more of the examples disclosed herein, when the program is executed on a processor, computer or computer network. As used herein, a computer program product refers to the program as a tradable product. The product may generally exist in an arbitrary format, such as in a paper format, or on a computer-readable data carrier. Specifically, the computer program product may be distributed over a data network.

With a further example the injection device comprises a dose dial and a trigger, e.g. provided at a proximal longitudinal end of the injection device. The injection device may be implemented as a pen-type injector. The dose dial and/or the trigger may be provided at a proximal longitudinal end of the pen-type injector. Here, the machine-readable identification may be provided in or on the dose dial and/or the trigger. The add-on device may be configured for releasable or detachable fastening to the proximal end of the injection device.

The add-on device, in particular its device body may comprise a receptacle to fit onto the dose dial and/or trigger. By implementing or arranging the machine-readable identification in or on the trigger or dose dial a geometric distance between the machine-readable identification and a reader or transceiver of the add-on device for readout of the machine-readable identification can be reduced to a minimum thus allowing to implement the transceiver or reader of the add-on device as a wireless near field transceiver operable to read a respective near field communication tag of the machine-readable identification of the device. This way, readout of the machine-readable identifier can only take place when the add-on device is correctly mounted to the proximal end of the injection device.

According to a further example the injection system comprises an external electronic device, e.g. implemented as a smartphone, a smart watch or as a tablet computer. The external electronic device is operable to read the machine-readable identification of the injection device. The external electronic device is further operable to transmit the identification of the injection device to the add-on device.

This way, the add-on device may be void of an own reader or transceiver operable to read the machine-readable identification of the injection device. Rather, it may be sufficient when the add-on device is equipped with only one transceiver, such as a wireless local range transceiver operable to wirelessly communicate with the external electronic device. Readout of the machine-readable identification of the injection device may be then provided and conducted through the external electronic device, which, when suitably paired with the add-on device is operable to provide the identifying information of the injection device to the add-on device, which then in turn may conduct a configuration or reconfiguration of the user perceptible device identification.

With this example the machine-readable identification of the injection device can be implemented in a variety of different ways. Here, the machine-readable identification may be implemented as a physical, mechanical or optical code, which is readable by a respective sensing arrangement and/or imaging system of the external electronic device.

Generally, the scope of the present disclosure is defined by the content of the claims. The disclosure is not limited to specific embodiments or examples but comprises any combination of elements of different embodiments or examples. Insofar, the present disclosure covers any combination of claims and any technically feasible combination of the features disclosed in connection with different examples or embodiments.

In the present context the term ‘distal’ or ‘distal end’ relates to an end of the injection device that faces towards an injection site of a person or of an animal. The term ‘proximal’ or ‘proximal end’ relates to an opposite end of the injection device, which is furthest away from an injection site of a person or of an animal.

The terms “drug” or “medicament” are used synonymously herein and describe a pharmaceutical formulation containing one or more active pharmaceutical ingredients or pharmaceutically acceptable salts or solvates thereof, and optionally a pharmaceutically acceptable carrier. An active pharmaceutical ingredient (“API”), in the broadest terms, is a chemical structure that has a biological effect on humans or animals. In pharmacology, a drug or medicament is used in the treatment, cure, prevention, or diagnosis of disease or used to otherwise enhance physical or mental well-being. A drug or medicament may be used for a limited duration, or on a regular basis for chronic disorders.

As described below, a drug or medicament can include at least one API, or combinations thereof, in various types of formulations, for the treatment of one or more diseases. Examples of API may include small molecules having a molecular weight of 500 Da or less; polypeptides, peptides and proteins (e.g., hormones, growth factors, antibodies, antibody fragments, and enzymes); carbohydrates and polysaccharides; and nucleic acids, double or single stranded DNA (including naked and cDNA), RNA, antisense nucleic acids such as antisense DNA and RNA, small interfering RNA (siRNA), ribozymes, genes, and oligonucleotides. Nucleic acids may be incorporated into molecular delivery systems such as vectors, plasmids, or liposomes. Mixtures of one or more drugs are also contemplated.

The drug or medicament may be contained in a primary package or “drug container” adapted for use with a drug delivery device. The drug container may be, e.g., a cartridge, syringe, reservoir, or other solid or flexible vessel configured to provide a suitable chamber for storage (e.g., short-or long-term storage) of one or more drugs. For example, in some instances, the chamber may be designed to store a drug for at least one day (e.g., 1 to at least 30 days). In some instances, the chamber may be designed to store a drug for about 1 month to about 2 years. Storage may occur at room temperature (e.g., about 20° C.), or refrigerated temperatures (e.g., from about −4° C. to about 4° C.). In some instances, the drug container may be or may include a dual-chamber cartridge configured to store two or more components of the pharmaceutical formulation to-be-administered (e.g., an API and a diluent, or two different drugs) separately, one in each chamber. In such instances, the two chambers of the dual-chamber cartridge may be configured to allow mixing between the two or more components prior to and/or during dispensing into the human or animal body. For example, the two chambers may be configured such that they are in fluid communication with each other (e.g., by way of a conduit between the two chambers) and allow mixing of the two components when desired by a user prior to dispensing. Alternatively or in addition, the two chambers may be configured to allow mixing as the components are being dispensed into the human or animal body.

The drugs or medicaments contained in the drug delivery devices as described herein can be used for the treatment and/or prophylaxis of many different types of medical disorders. Examples of disorders include, e.g., diabetes mellitus or complications associated with diabetes mellitus such as diabetic retinopathy, thromboembolism disorders such as deep vein or pulmonary thromboembolism. Further examples of disorders are acute coronary syndrome (ACS), angina, myocardial infarction, cancer, macular degeneration, inflammation, hay fever, atherosclerosis and/or rheumatoid arthritis. Examples of APIs and drugs are those as described in handbooks such as Rote Liste 2014, for example, without limitation, main groups 12 (anti-diabetic drugs) or 86 (oncology drugs), and Merck Index, 15th edition.

Examples of APIs for the treatment and/or prophylaxis of type 1 or type 2 diabetes mellitus or complications associated with type 1 or type 2 diabetes mellitus include an insulin, e.g., human insulin, or a human insulin analogue or derivative, a glucagon-like peptide (GLP-1), GLP-1 analogues or GLP-1 receptor agonists, or an analogue or derivative thereof, a dipeptidyl peptidase-4 (DPP4) inhibitor, or a pharmaceutically acceptable salt or solvate thereof, or any mixture thereof. As used herein, the terms “analogue” and “derivative” refers to a polypeptide which has a molecular structure which formally can be derived from the structure of a naturally occurring peptide, for example that of human insulin, by deleting and/or exchanging at least one amino acid residue occurring in the naturally occurring peptide and/or by adding at least one amino acid residue. The added and/or exchanged amino acid residue can either be codable amino acid residues or other naturally occurring residues or purely synthetic amino acid residues. Insulin analogues are also referred to as “insulin receptor ligands”. In particular, the term “derivative” refers to a polypeptide which has a molecular structure which formally can be derived from the structure of a naturally occurring peptide, for example that of human insulin, in which one or more organic substituent (e.g. a fatty acid) is bound to one or more of the amino acids. Optionally, one or more amino acids occurring in the naturally occurring peptide may have been deleted and/or replaced by other amino acids, including non-codeable amino acids, or amino acids, including non-codeable, have been added to the naturally occurring peptide.

Examples of insulin analogues are Gly(A21), Arg(B31), Arg(B32) human insulin (insulin glargine); Lys(B3), Glu(B29) human insulin (insulin glulisine); Lys(B28), Pro(B29) human insulin (insulin lispro); Asp(B28) human insulin (insulin aspart); human insulin, wherein proline in position B28 is replaced by Asp, Lys, Leu, Val or Ala and wherein in position B29 Lys may be replaced by Pro; Ala(B26) human insulin; Des(B28-B30) human insulin; Des(B27) human insulin and Des(B30) human insulin.

Examples of insulin derivatives are, for example, B29-N-myristoyl-des(B30) human insulin, Lys(B29) (N-tetradecanoyl)-des(B30) human insulin (insulin detemir, Levemir®); B29-N-palmitoyl-des(B30) human insulin; B29-N-myristoyl human insulin; B29-N-palmitoyl human insulin; B28-N-myristoyl LysB28ProB29 human insulin; B28-N-palmitoyl-LysB28ProB29 human insulin; B30-N-myristoyl-ThrB29LysB30 human insulin; B30-N-palmitoyl-ThrB29LysB30 human insulin; B29-N-(N-palmitoyl-gamma-glutamyl)-des(B30) human insulin, B29-N-omega-carboxypentadecanoyl-gamma-L-glutamyl-des(B30) human insulin (insulin degludec, Tresiba®); B29-N-(N-lithocholyl-gamma-glutamyl)-des(B30) human insulin; B29-N-(ω-carboxyheptadecanoyl)-des(B30) human insulin and B29-N-(ω-carboxyheptadecanoyl) human insulin.

Examples of GLP-1, GLP-1 analogues and GLP-1 receptor agonists are, for example, Lixisenatide (Lyxumia®), Exenatide (Exendin-4, Byetta®, Bydureon®, a 39 amino acid peptide which is produced by the salivary glands of the Gila monster), Liraglutide (Victoza®), Semaglutide, Taspoglutide, Albiglutide (Syncria®), Dulaglutide (Trulicity®), rExendin-4, CJC-1134-PC, PB-1023, TTP-054, Langlenatide/HM-11260C (Efpeglenatide), HM-15211, CM-3, GLP-1 Eligen, ORMD-0901, NN-9423, NN-9709, NN-9924, NN-9926, NN-9927, Nodexen, Viador-GLP-1, CVX-096, ZYOG-1, ZYD-1, GSK-2374697, DA-3091, MAR-701, MAR709, ZP-2929, ZP-3022, ZP-DI-70, TT-401 (Pegapamodtide), BHM-034. MOD-6030, CAM-2036, DA-15864, ARI-2651, ARI-2255, Tirzepatide (LY3298176), Bamadutide (SAR425899), Exenatide-XTEN and Glucagon-Xten.

An example of an oligonucleotide is, for example: mipomersen sodium (Kynamro®), a cholesterol-reducing antisense therapeutic for the treatment of familial hypercholesterolemia or RG012 for the treatment of Alport syndrom. Examples of DPP4 inhibitors are Linagliptin, Vildagliptin, Sitagliptin, Denagliptin, Saxagliptin, Berberine.

Examples of hormones include hypophysis hormones or hypothalamus hormones or regulatory active peptides and their antagonists, such as Gonadotropine (Follitropin, Lutropin, Choriongonadotropin, Menotropin), Somatropine (Somatropin), Desmopressin, Terlipressin, Gonadorelin, Triptorelin, Leuprorelin, Buserelin, Nafarelin, and Goserelin.

Examples of polysaccharides include a glucosaminoglycane, a hyaluronic acid, a heparin, a low molecular weight heparin or an ultra-low molecular weight heparin or a derivative thereof, or a sulphated polysaccharide, e.g. a poly-sulphated form of the above-mentioned polysaccharides, and/or a pharmaceutically acceptable salt thereof. An example of a pharmaceutically acceptable salt of a poly-sulphated low molecular weight heparin is enoxaparin sodium. An example of a hyaluronic acid derivative is Hylan G-F 20 (Synvisc®), a sodium hyaluronate.

The term “antibody”, as used herein, refers to an immunoglobulin molecule or an antigen-binding portion thereof. Examples of antigen-binding portions of immunoglobulin molecules include F(ab) and F(ab′)2 fragments, which retain the ability to bind antigen. The antibody can be polyclonal, monoclonal, recombinant, chimeric, de-immunized or humanized, fully human, non-human, (e.g., murine), or single chain antibody. In some embodiments, the antibody has effector function and can fix complement. In some embodiments, the antibody has reduced or no ability to bind an Fc receptor. For example, the antibody can be an isotype or subtype, an antibody fragment or mutant, which does not support binding to an Fc receptor, e.g., it has a mutagenized or deleted Fc receptor binding region. The term antibody also includes an antigen-binding molecule based on tetravalent bispecific tandem immunoglobulins (TBTI) and/or a dual variable region antibody-like binding protein having cross-over binding region orientation (CODV).

The terms “fragment” or “antibody fragment” refer to a polypeptide derived from an antibody polypeptide molecule (e.g., an antibody heavy and/or light chain polypeptide) that does not comprise a full-length antibody polypeptide, but that still comprises at least a portion of a full-length antibody polypeptide that is capable of binding to an antigen. Antibody fragments can comprise a cleaved portion of a full length antibody polypeptide, although the term is not limited to such cleaved fragments. Antibody fragments that are useful in the present invention include, for example, Fab fragments, F(ab′)2 fragments, scFv (single-chain Fv) fragments, linear antibodies, monospecific or multispecific antibody fragments such as bispecific, trispecific, tetraspecific and multispecific antibodies (e.g., diabodies, triabodies, tetrabodies), monovalent or multivalent antibody fragments such as bivalent, trivalent, tetravalent and multivalent antibodies, minibodies, chelating recombinant antibodies, tribodies or bibodies, intrabodies, nanobodies, small modular immunopharmaceuticals (SMIP), binding-domain immunoglobulin fusion proteins, camelized antibodies, and VHH containing antibodies. Additional examples of antigen-binding antibody fragments are known in the art.

The terms “Complementarity-determining region” or “CDR” refer to short polypeptide sequences within the variable region of both heavy and light chain polypeptides that are primarily responsible for mediating specific antigen recognition. The term “framework region” refers to amino acid sequences within the variable region of both heavy and light chain polypeptides that are not CDR sequences, and are primarily responsible for maintaining correct positioning of the CDR sequences to permit antigen binding. Although the framework regions themselves typically do not directly participate in antigen binding, as is known in the art, certain residues within the framework regions of certain antibodies can directly participate in antigen binding or can affect the ability of one or more amino acids in CDRs to interact with antigen. Examples of antibodies are anti PCSK-9 mAb (e.g., Alirocumab), anti IL-6 mAb (e.g., Sarilumab), and anti IL-4 mAb (e.g., Dupilumab).

Pharmaceutically acceptable salts of any API described herein are also contemplated for use in a drug or medicament in a drug delivery device. Pharmaceutically acceptable salts are for example acid addition salts and basic salts.

Those of skill in the art will understand that modifications (additions and/or removals) of various components of the APIs, formulations, apparatuses, methods, systems and embodiments described herein may be made without departing from the full scope and spirit of the present invention, which encompass such modifications and any and all equivalents thereof.

An example drug delivery device may involve a needle-based injection system as described in Table 1 of section 5.2 of ISO 11608-1:2014(E). As described in ISO 11608-1:2014(E), needle-based injection systems may be broadly distinguished into multi-dose container systems and single-dose (with partial or full evacuation) container systems. The container may be a replaceable container or an integrated non-replaceable container.

As further described in ISO 11608-1:2014(E), a multi-dose container system may involve a needle-based injection device with a replaceable container. In such a system, each container holds multiple doses, the size of which may be fixed or variable (pre-set by the user). Another multi-dose container system may involve a needle-based injection device with an integrated non-replaceable container. In such a system, each container holds multiple doses, the size of which may be fixed or variable (pre-set by the user).

As further described in ISO 11608-1:2014(E), a single-dose container system may involve a needle-based injection device with a replaceable container. In one example for such a system, each container holds a single dose, whereby the entire deliverable volume is expelled (full evacuation). In a further example, each container holds a single dose, whereby a portion of the deliverable volume is expelled (partial evacuation). As also described in ISO 11608-1:2014(E), a single-dose container system may involve a needle-based injection device with an integrated non-replaceable container. In one example for such a system, each container holds a single dose, whereby the entire deliverable volume is expelled (full evacuation). In a further example, each container holds a single dose, whereby a portion of the deliverable volume is expelled (partial evacuation).

### BRIEF DESCRIPTION OF THE DRAWINGS

In the following, numerous examples of a data logging device for monitoring use of an injection device as well as a respective injection device will be described in greater detail by making reference to the drawings, in which:

FIG. 1 schematically illustrates an example of an injection device,

FIG. 2 shows the process of assembling an add-on device to a proximal end of the injection device,

FIG. 3 schematically illustrates a cross-section of an example of the injection device,

FIG. 4 shows a configuration of an injection system including the injection device, the add-on device and at least one external electronic device

FIG. 5 shows a pairing between the external electronic device and the add-on device,

FIG. 6 shows the process of mounting the add-on device to the injection device

FIG. 7 shows a configuration of an external electronic device upon successful readout of machine-readable identification of the injection device,

FIG. 8 shows a configuration of an injection system,

FIG. 9 shows a configuration of another injection system

FIG. 10a, b show a further example of an add-on device,

FIG. 11 shows a block diagram of the add-on device, the injection device and the external electronic device,

FIG. 12 shows a block diagram of the electronic circuit of the machine-readable identification,

FIG. 13 shows an example of the electronic circuit on a substrate,

FIG. 14 shows a flowchart of a method of configuring the add-on device.

### DETAILED DESCRIPTION

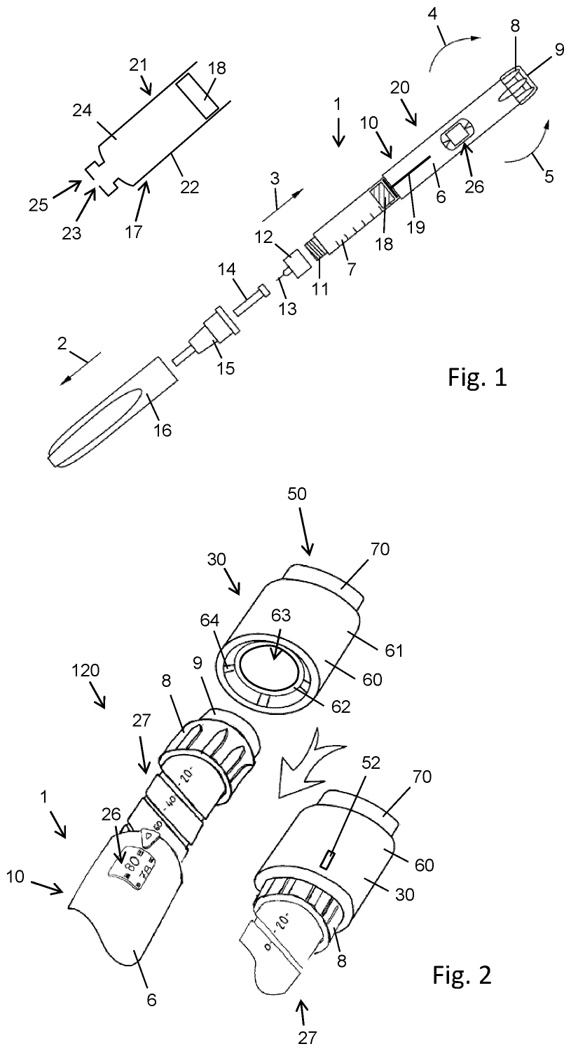

FIG. 1 shows an example of a drug delivery device, which is implemented as a handheld injection device 1. The injection device 1 may comprise or may be implemented as a pen-type injector. It may be implemented as a disposable injection device or as a reusable injection device. With some examples the injection device 1 is implemented as an autoinjector. The injection device 1 is of elongated shape. It may extend along a longitudinal direction. Towards a longitudinal distal direction 2 the drug delivery device 1 comprises a dispensing end for dispensing or injecting the medicament 24. Towards the proximal direction 3 the injection device 1 comprises at least one of a dose member 8 and a trigger 9, by way of which a dose of equal or individual or different size can be set and dispensed, respectively.

The injection device 1 comprises a housing 10. The housing 10 may comprise numerous housing components, such as a body 6 and a container part 7, e.g. implemented as a cartridge holder 7. The body 6 may be sized and configured to accommodate a drive mechanism 20. The container part 7 is sized and configured to accommodate a medicament container 21, e.g. implemented as a cartridge containing the liquid medicament 24. The medicament container 21 comprises a tubular-shaped barrel 22 sealed towards the distal end by a seal 23. The seal 23 may comprise a pierceable septum fixed to an outlet 25 of the medicament container 21. Towards a proximal end the interior of the barrel 22 is sealed by a piston 18 or stopper, which is slidably disposed inside the barrel 22.

By advancing the piston 18 towards the distal direction 2 a dose of the medicament 24 can be expelled from the medicament container 21. In use the medicament container 21 is arranged inside the container part 7. The drive mechanism 20 of the injection device 1 comprises a piston rod 19, which is displaceable in distal direction 2 for advancing the piston 18 towards the outlet 25 of the medicament container 21. Details of the drive mechanism are not further illustrated and described here. With some examples, the drive mechanism 20 may be implemented as an all-mechanical drive mechanism, where a user has to provide an entirety of a dispensing force required to move the piston rod 19 and hence the piston 18 in distal direction 2. With other examples, the drive mechanism comprises a mechanical energy storage configured to provide at least a portion of the dispensing force. Examples of drive mechanisms can be found e.g. in WO2004/078241 A1, WO 2014/033197 A1 or WO 2014/033195 A1 the entirety of which are herein incorporated by reference.

With some examples, as e.g. described or shown in FIG. 2 the injection device 1 and hence the drive mechanism 20 may comprise a dial extension 27, which projects and moves in proximal direction 3 from a proximal end of the body 6 when or during setting a dose and which returns into its initial distal end position during a dose injection procedure. For this, a user may use a thumb 114 of his hand 110 to exert a distally directed pressure onto the trigger 9 thereby urging the dial extension 27 in distal direction 2 during and/or for a dose injection procedure.

For setting or dialing of a dose a user may twist or rotate the dose dial 8, e.g. in a dose incrementing direction 4, hence in a clockwise sense as seen from the proximal end. For correcting a dose previously set the user may also rotate the dose dial 8 in an opposite dose decrementing direction 5. The size of the dose is typically illustrated in a window 26 provided in or on the body 6 of the injection device 1. Prior to inject a dose of the medicament 24 the distal end of the container part 7 or cartridge holder has to be connected with a needle assembly 12. For this, the distal end of the cartridge holder 7 comprises a connector 11, e.g. in form of a threaded interface to engage with a complementary shaped threaded counter interface of the needle assembly 12.

The needle assembly 12 is detachably or releasably fixable to the container part 7. It comprises a double-tipped injection needle 13. A proximal end of the injection needle (not shown) is configured to enter into a through opening at the distal end face of the connector 11 or container part 7 so as to pierce or to penetrate the seal 23 of the medicament container 21. The distal end of the injection needle 13 is typically covered by a detachable inner needle cap 14. The entirety of the needle assembly 12 may be covered by a detachable outer needle cap 15.

The container part 7 and hence a portion of the housing 10 is to be received in a protective cap 16, which is detachably connectable to the cartridge holder 7 or body 6.

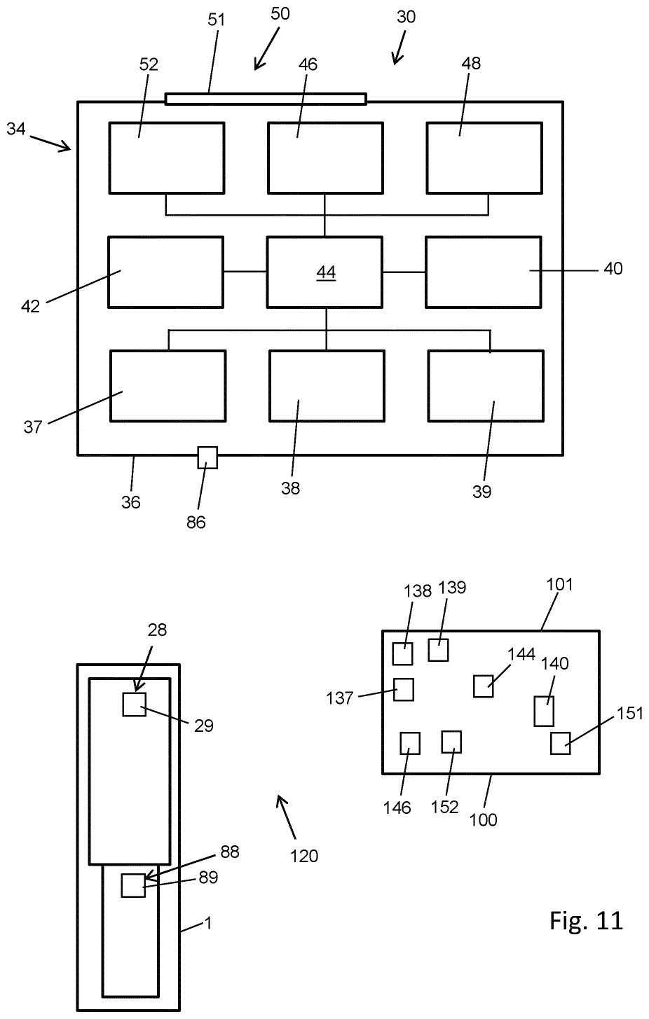

In FIGS. 2 and 4 there is shown an example of an add-on device 30 configured for fastening to the proximal end of the injection device 1. The add-on device 30 comprises a sensor 48 or sensor assembly, which is operable to detect, to recognize, to characterize and/or to measure an operation of the injection device 1. Typically, the sensor 48 of the add-on device 30 and as indicated in the block diagram of FIG. 11 is capable or operable to quantitatively determine or to measure at least one of a position and a movement of a movable component 80, 81, 82, 83 of the drive mechanism 20 of the injection device 1. The movable component 80, 81, 82, 83 is movable relative to at least one of the body 6 and the medicament container 21 during or for setting and/or injecting of a dose of the medicament 24.

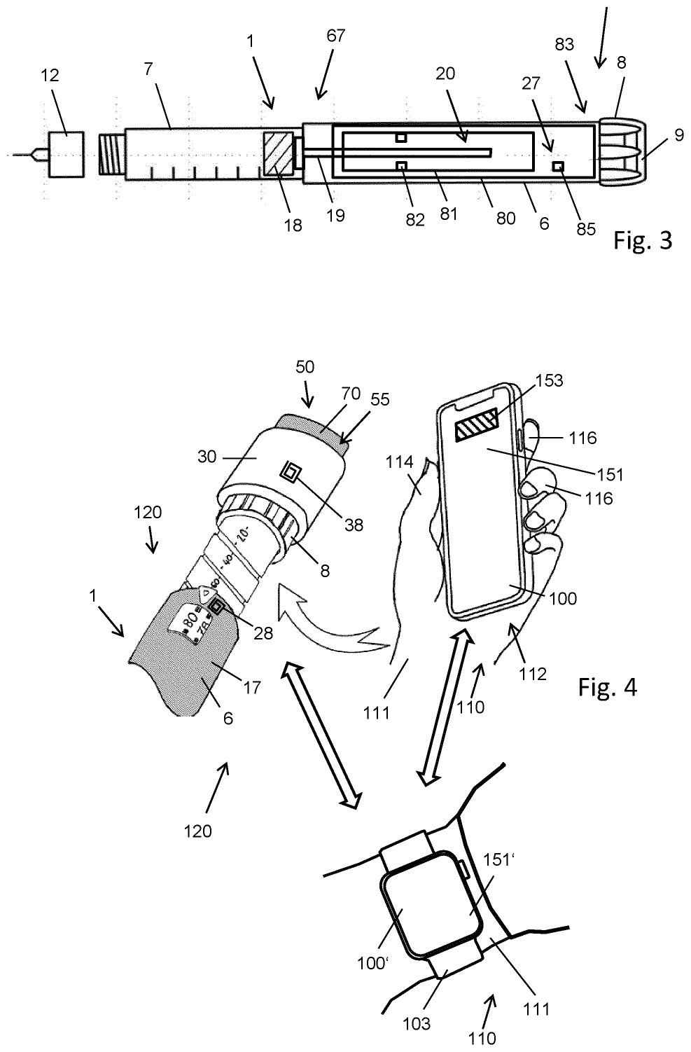

This movement or position is quantitatively measurable by the sensor 48. Typically, the sensor 48 is implemented as an electronic sensor. It is capable to detect or to measure a degree of longitudinal translation and/or rotation of the at least one movable component 80, 81, 82, 83 relative to the housing 10 or relative to the device body 60 when the add-on device 30 is attached to the injection device 1. Examples of movable components 80 are schematically illustrated in the cross-section of the injection device 1 according to FIG. 3

The sensor 48 of the add-on device 30 is capable or operable to generate a sensor signal or a sequence of sensor signals, which due to the coupling or connection to the module processor 44 can be processed by the module processor 44 in order to derive or to calculate a size of a dose currently set or dispensed.

With some examples the movable component 80 is a dose setting member or a dose setting sleeve. With further examples the movable component 81 is implemented as a drive member or drive sleeve 81 of the drive mechanism 20. With further examples the movable component is implemented as a volume indicator 82, e.g. as a single dose indicating member, whose position or configuration relative to the body 6 is directly indicative of the size of a dose currently set. With further examples the movable component 82, hence the volume indicator is a last dose indicating member, the position or configuration of which relative to the body 6 is indicative of a remaining amount of medicament left in the cartridge or medicament container 21.

With some examples the movable component 83 is or comprises an encoding, such as a digital encoding, which is movable relative to the sensor 48 such that the sensor is capable to count a number of discrete code portions of the encoding so as to gather respective quantitative movement information of the movable component 80. Generally, there may be provided different types of spatial encodings, such as an optical or visual encoding, an electrical or electrostatic encoding a magnetic encoding or mechanical encoding. Depending on the type of an encoding provided on or inside the movable component 80, 81, 82, 83 the sensor 48 is correspondingly configured. Hence, the sensor 48 may comprise at least one of an optical sensor, an electrical or electrostatic sensor and a magnetic sensor or mechanical sensor, such as a micromechanical switch, each of which being operable to quantitatively measure a relative position or relative movement of the movable component 80, 81, 82, 83.

With some examples the movable component 80, 81, 82, 83 is subject to a longitudinal and/or rotational movement relative to the body 6, relative to the medicament container 21 and/or relative to the device body 60 during at least one of setting of the dose and dispensing of the dose. Typically, the encoding as provided on or with the movable component 80, 81, 82, 83 is encoded along the direction of movement of the movable component relative to the sensor 48 so as to enable a respective quantitative measurement of the respective dose setting or dose dispensing movement.

As further indicated in FIG. 3 the drive mechanism 20 may be optionally equipped with a locking mechanism 85, which is operable by a locking controller of the add-on device 30 when the add-on device 30 is correctly assembled to the injection device 1. The locking mechanism 85 is operable to block or to lock the drive mechanism 20 so as to prevent unauthorized or unintended setting of a dose and/or dispensing or injecting of the dose. The locking mechanism 85 is controllable by the locking controller of the add-on device. The locking mechanism 85 may be implemented electromechanically and may be electrically or electronically controlled by the locking controller 86 of the add-on device 30. This way, the add-on device 30 may block an unauthorized or unintended use of the injection device 1.

The add-on devices 30 as illustrated in the various FIGS. 2-10 is detachably connectable to the dose dial 8. It comprises a device body 60 with a tubular-shaped sidewall 61. Towards the distal end the sidewall 61 confines a receptacle 63, which is sized to receive the dose dial 8 and the trigger 9 of the injection device 1. For this, the inside of the sidewall 60 may comprise one or numerous fastening ribs 64, which are configured to provide a slip free fastening of the add-on device 30 to the dose dial 8.

The device body 60 comprises the receptacle 63 at a distal end section of the add-on device 30. The receptacle 63 is open towards the distal direction 2. It is sized and configured to fit onto the proximal end of the injection device 1. An inside surface of the sidewall 61 confining the receptacle 63 comprises numerous fastening ribs 64, e.g. of an elastic or elastically deformable material, such as an elastomeric material.

The fastening ribs 64 provide a friction fit with the dose dial 8 when the device body 60 is assembled to the dose dial 8 of the injection device 1. The receptacle 63 may be confined in longitudinal direction by a flange portion 62 protruding radially inwardly from the tubular-shaped sidewall 61 of the receptacle 63. The flange portion 62 is located proximally offset from the insert opening of the receptacle 63. The flange portion 62 may be of annular shape and may be configured to axially abut on a stepped down section at the proximal end of the dose dial 8. Hence, the trigger 9, which comprises a reduced diameter compared to the dose dial 8 may protrude in proximal direction 3 through the flange portion 62 while the flange portion 62 may rest or abut against the proximal end face of the dose dial 8 when the add-on device 30 is suitably fastened to the dose dial 8.

The add-on device 30 further comprises a movable part 70 protruding in proximal direction from the device body 60. The movable part 70 comprises or forms an auxiliary trigger or trigger button, which is configured to mechanically engage with the trigger 9 of the injection device 1 when the add-on device 30 is correctly assembled to the injection device 1.

Typically, the add-on device 30, in particular, the device body 60 is frictionally engageable with the dose dial 8. In this way, a user may apply a dose setting torque onto the dose dial via the device body 60. Instead of rotating the dose dial 8 for setting of a dose the user may simply rotate the device body 60 relative to the body 6 of the injection device 1. This way, the dial extension 27 may become subject to a dose incrementing dialing or rotating motion. A respective size of a dose currently set will then be displayed in the window 26 of the body 6 of the injection device 1 as illustrated in FIG. 2.

For dispensing of a dose the user has to depress the movable part 70, which may then be subject to a distally directed motion relative to the device body 60. The movable part 70, which may be in direct or indirect mechanical engagement with the trigger 9, may then apply a respective dispensing force onto the trigger 9 thereby initiating a dose dispensing action of the injection device 1.

The add-on device 30 as schematically illustrated in FIG. 11 comprises an electronic module 34. The electronic module 34 comprises a printed circuit board 36. The electronic module 34 comprises a module processor 44, an electronic and hence digital module memory 40 and a clock 42. Furthermore, the electronic module 34 comprises a power source 46 and the sensor 48. The electronic module 34 also comprises a signal generator 52 coupled to the processor 44 and/or coupled to the power source 46. The electronic module 34 further comprises at least one of a reader 37 and a wireless transceiver 38, 39. The add-on device 30 may also comprise a user perceptible device identification 50.

With some examples the device identification 50 comprises or includes the signal generator 52 coupled to the module processor 44 and operable or reconfigurable by the module processor 44. With some examples the device identification 50 may be indicative of the injection device 1 to which the add-on device 30 is actually connected or coupled to.

With some examples the signal generator 52 is operable to generate at least one of a visual, an acoustic and a haptic signal or device identification and is hence operable to produce or to generate a visual identifier, an acoustic identifier or a haptic identifier or a respective alert signal. With some examples the add-on device 30 comprises numerous signal generators 52 of equal or different type, e.g. optical, acoustic or haptic type.

With the example of FIG. 2 the signal generator 52 may comprise a speaker operable to generate an acoustic signal, wherein the acoustic signal may represent or constitute an alert signal e.g. in form of an acoustic code or sequence being characteristic for a specific configuration or setting of the injection device 1 or add-on device 30.

FIG. 4 is illustrative of an injection system 120 comprising at least the add-on device 30 and the injection device 1. Optionally, the injection system 120 comprises one or several external electronic devices 100, 101′. The external electronic device 100 is implemented as a smart phone comprising a housing 101 and being equipped with a device display 151 to visually illustrate various types of information, such as various illustrations 153 or notifications 154 to a user. The display 151 may be implemented as a touch sensitive display. The device display 151 may be operable to provide or to emulate a device signal generator 152. The device display 151 is operable to provide at least one of an illustration 153 and a notification 154 to a user.

The further optional external electronic device 100′ is implemented as a smartwatch comprising a respective housing and a display 151′ and further comprising a wristband 103 for fixing the external electronic device 100′ to the hand 110 or wrist 111 of a user. Like the external electronic device 100 also the external electronic device 100′ is operable to provide at least one of an illustration 153 and a notification 154 to the user. Generally, the external electronic device 100, 100′ may be equipped with a separate device signal generator 152, such as a speaker or a vibration module by way of which a user perceivable signal can be generated so as to provide an alert signal to a user of the respective device.

The electronic device 100 is typically implemented as a handheld electronic device. It can be held in a hand 110 of a user or may be worn on the wrist 111 of a user. With the example as illustrated in FIG. 4 the electronic device 100 is held in a palm 112 of the user. The device display 151 is implemented as a touch sensitive display and can be operated by a thumb 114 and/or by various fingers 116 of the user's hand 110.

The electronic module 34 of the add-on device 30 as schematically illustrated in FIG. 11 comprises at least one of a reader 37 and a transceiver 38, 39 operable for wireless communication with at least one of a machine-readable identification 28, 88 of the injection device 1 or with a complementary or correspondingly implemented device reader 137 and/or device transceiver 138, 139 of the external electronic device 100, 100′.

With typical examples the transceivers 38, 138 are implemented as radiofrequency transceivers of near field type. The transceivers 38, 138 may be implemented as wireless short range transceivers or near field transceivers. They may be implemented as a so-called NFC transceivers allowing for wireless communication within a limited spatial range of a few centimeters or decimeters. The transceivers 38, 138 may be implemented as active and/or passive NFC tags or readers. Typically, the transceiver 38 is implemented as a passive NFC tag and the transceiver 138 is implemented as an active NFC tag or NFC reader.

The transceivers 39, 139 may be implemented as wireless local range transceivers, such as RFID, Bluetooth, Bluetooth low energy (BLE) or UWB transceivers. Also here, the transceiver 39 may be implemented as a passive communication tag whereas the transceiver 139 may be implemented as an active transceiver, hence as a reader. Typically, the transmission range of the transceivers 39, 139 is larger than the transmission range of the transceivers 38, 138. The transmission range of the transceivers 39, 139 may be in a range of several meters or decameters.

The optional reader 37, 137 may be implemented as an optical reader or optical information gathering unit, e.g. comprising an imaging system so as to capture or to record a visual or optical machine-readable identification 28, 88 as provided on or inside the injection device 1.

The external electronic device 100, 100′ typically comprises a device processor 144 coupled to an electronic device memory 140. The electronic device 100 further comprises a device power source 146. Also, the electronic device 100 comprises a device display 151, typically implemented as a touch sensitive display. The device display 151 may be operable to provide at least one of an illustration 153 and a notification 154 to a user.

The device transceiver 138 is operable to communicate with the transceiver 38 of the add-on device. The transceiver 139 is configured to communicate with the transceiver 39 of the add-on device 30. The reader 137 may be operable to read a visual identifier as provided on the injection device 1. With some examples the device reader 137 comprises an imaging system. It may comprise a camera objective and a spatially resolving detector in order to read or to capture a visual code, e.g. provided on an outside surface of the injection device 1 or as provided on an outside surface of the medicament container 21.

The external device 100 is configured to set up a communication link, e.g. a wireless communication link with the add-on device 30. For this, the local range transceiver 39 of the add-on device 30 may communicate wirelessly with the local range device transceiver 139 of the add-on device 100. Once a communication link has been established between the electronic device 100 and the add-on device 30 the electronic device 100 may be operable to visibly illustrate information or data of the injection device or medicament container 21 on the device display 151.

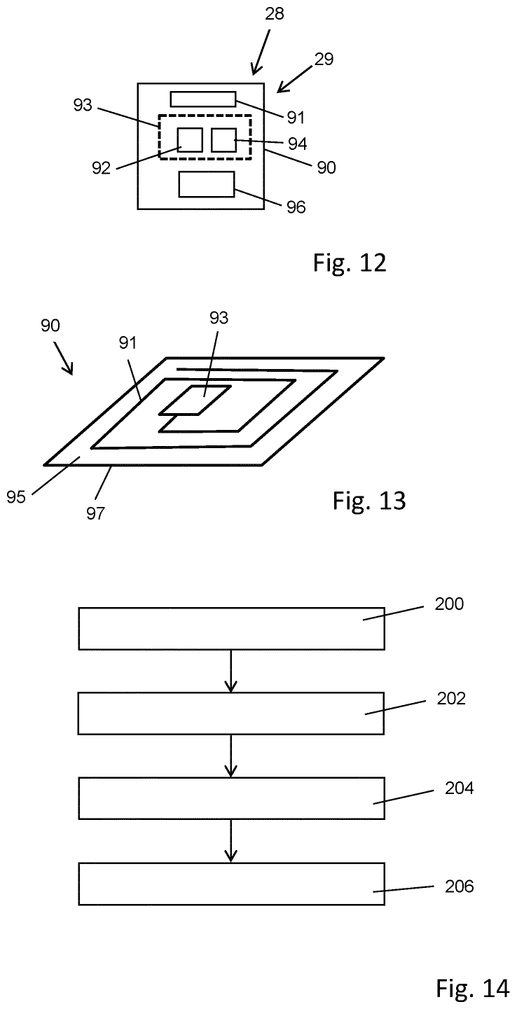

The injection device 1 is provided with a machine-readable identification 28, 88 as schematically illustrated in FIG. 11. The machine-readable identification 28 comprises an electronic identifier 29. The machine-readable identification 88 comprises an electronic identifier 89. The electronic identifier 28 may comprise a passive wireless communication tag as schematically illustrated in FIG. 12.

As illustrated in FIG. 13 the electronic identifier 29 may comprise an electronic circuit 90 featuring a wireless communication antenna 91 and an integrated circuit 93. The integrated circuit 93 comprises an electronic and hence digital memory 92 to and a processor 94 connected to the memory 92. Of course, the integrated circuit 93 and hence the processor 94 is connected to the antenna 91. The electronic identifier 29 may optionally comprise a power source 96 in order to provide electrical power or energy for the processor 94. The electronic circuit 90 may be provided on a planar substrate 95. The substrate 95 may be pliable or foldable. It may comprise a flexible structure or foil that allows for an easy mounting, fastening e.g. adhesive fastening, of the substrate 94 and hence of the entire electronic identifier 29 to at least one of the tubular shaped body 6, the container part 7 or the medicament container 21.

The electronic circuit 90 may be a printed electronic circuit, which is printed on the substrate 95. Here, the substrate 95 may comprise or constitute an e.g. flexible printed circuit board. The substrate 95 may be further provided with an adhesive layer 97 that allows for an easy and straightforward adhesive attachment of the machine-readable identification 28, 88 to a predefined portion on or inside the injection device 1. The electronic identifier 89 may comprise an identical or similar structure as the electronic identifier 29 as shown in FIGS. 12 and 13.

With some examples the machine-readable identification 28, 88 is implemented in a label 17 located on one of the body 6, the container part 7 or the medicament container 21. The label 17 comprises or contains the machine-readable identification 28, 88. With some examples the label 17 comprises a passive radiofrequency tag. With further examples the label 17 comprises machine-readable identifications 28, 88 implemented as a visual or optical code that can be captured by a reader 37 or device reader 137, which is implemented as or comprises an imaging system.

At least one of the reader 37, the device reader 137 or at least one of the transceivers 38, 39 or device transceivers 138, 139 is or are operable to read the machine-readable identification 28, 88 thereby obtaining at least one of a device information and a container information e.g. stored in the memory 92 of the electronic circuit 90.

Typically, the machine-readable identification 28 is provided or located on or inside the body 6 of the housing 10 of the injection device 1. The further machine-readable identification 88 is provided on or inside the container part 7. It may be provided on or inside the medicament container 21. With some examples the machine-readable identification 28 may be provided in or on the piston 18 of the medicament container 21.

The machine-readable identification 28, 88 may be implemented as a passive RF communication tag, such as a NFC tag. The information, e.g. the device information and/or the container information stored in at least one of the machine-readable identification 28, 88 is readable by the electronic module 34 and hence by any one of the transceivers 38, 39 of the add-on device 30 and/or by any of the transceivers 138, 139 of the external electronic device 100, 100′.

With some examples and especially when the injection device 1 is implemented as a disposable injection device, which does not support replacement of the medicament container 21 it is sufficient when the injection device 1 comprises only one machine-readable identification 28. It may be of particular advantage when the machine-readable identification 28 is provided at or near a proximal end 68 of the body 6. It may be provided on or inside the dose dial 8 or the trigger 9 so as to be within the transmission range of the transceiver 38, 39 when the add-on device 30 is attached to the proximal end of the injection device 1.

With some examples the distal end 67 of the body 6 is detachably connectable to a proximal end of the container part 7. Then, the injection device 1 is implemented as a reusable device that allows a replacement of the container part 7 and/or a replacement of the medicament container 21 inside the container part 7. With reusable injection devices 1 it may be of particular benefit when both, the body 6 and the container part 7 are provided with a separate machine-readable identification 28, 88. As illustrated in FIG. 11, the container part 7 is provided with the machine-readable identification 88 whereas the body 6 is provided with the machine-readable identification 28.

As illustrated in FIG. 6 the transceiver 38 may be located inside the receptacle 63 of the device body 60 so as to minimize a spatial distance to the machine-readable identification 28 as provided at or near the proximal end 68 of the body 6. By making use of near field communication technology for the machine-readable a notification 28, 88 and the respective transceivers 38, 138 it can be ensured, that the machine-readable identification and the device information and/or container information stored therein can only be acquired or obtained when the add-on device 30 and/or the external electronic device 100, 100′ is within the near field transmission range.

Generally and when correctly assembled or attached to the injection device 1 the add-on device 30, in particular the electronic module 34, is capable to read at least one of a device information and a container information from the machine-readable identification 28, 88 as provided on or inside the injection device 1. Moreover, the module processor 44 is operable to receive and/or to process signals from the sensor 48 being indicative of a quantity of a movement of the movable component 80, 81, 82, 83 relative to at least one of the housing 10, the medicament container 21 and the device body 60 during operation of the injection device 1.

The electronic module 34 is operable to conduct or to execute an automated calibration procedure. For calculating or determining a size of a dose of the medicament currently set or dispensed by the injection device not only the sensor signal or sensor signals as obtained from the sensor 48 but also the device information and/or the container information as provided by the machine-readable identification 28, 88 are taken into account and are considered respectively.

Typically, the container information is indicative of at least one of a type of a medicament contained in the medicament container a concentration of a pharmaceutical substance of the medicament contained in the container, a manufacturing date of the medicament, a use by date of the medicament or, a LOT number of the medicament or medicament container and/or environmental parameters, such as temperature, humidity or radiation intensity to which the medicament container has been exposed. The container information may further include information about the type of the container in which the medicament is currently stored or contained. Insofar, the container information may contain container specific information, such as a type of a primary container, e.g. filled with the medicament or a type of a secondary container, e.g. a container part of an injection device, such as a cartridge holder configured to receive a primary container and configured for attachment to a body of a housing of an injection device.

Typically, the device information is indicative of at least one a type of an injection mechanism or drive mechanism of the injection device. The device information may thus characterize the dose setting and/or dose dispensing operability of the respective injection device. The device identification may further contain data or information about suitable medicaments or medicament containers to be exclusively used with the respective injection device.

In this way, the sensor signal can be concurrently processed with at least one of the container information and the device information in order to correctly calculate or to correctly determine the size of the dose currently set or dispensed by the injection device.

The container information or the device information may be required to correctly translate the sensor signals obtained from the sensor 48 into a size of a dose.

Typically and upon mounting the add-on device 30 to e.g. the proximal end 68 of the body 6 of the injection device 1 or when attaching the add-on device 30 to the dose dial 8 a readout of the machine-readable identification 28, 88 may be automatically triggered. For this, the add-on device 30 may be provided with a contact sensor, e.g. with a mechanical or electrical contact that is operable to detect the presence of e.g. the dose dial 8 inside the receptacle 63 of the device body 60. Moreover, a reading of at least one of the device information and the container information from the machine-readable identification 28 may be also triggered, e.g. when the transceiver 38 gets in close vicinity, i.e. in the transmission range to the machine-readable identification 28.

The device information or container information that is obtained by the transceiver 38 may be processed by the module processor 44 and may be stored in the module memory 40. This way, the electronic module 34 may be calibrated to the device specific device information and/or container information.

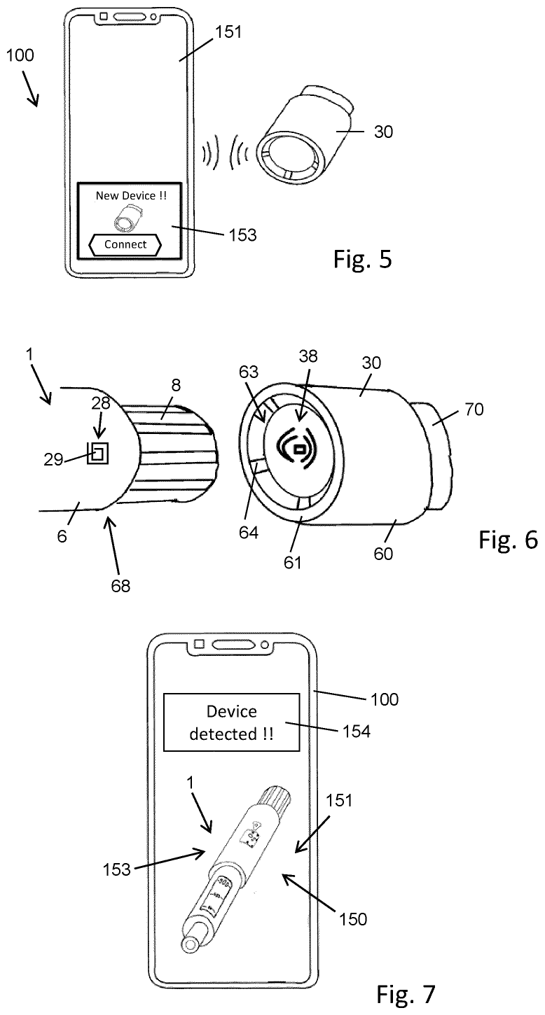

As further illustrated in FIG. 5, the add-on device 30 may optionally communicate with the external electronic device 100. It may set up a communication link, e.g. a wireless communication link when the external electronic device 100 and the add-on device 30 are within a predefined transmission range. Here, the add-on device 30 and the external electronic device 100 may communicate by the local range transceivers 39, 139.

In the course or after attaching or mounting the add-on device 30 to the injection device 1 as illustrated in FIG. 6 and when the device information and/or container information has been successfully obtained or read by the add-on device 30 the communication between the add-on device 30 and the external electronic device 100 may enable to illustrate or indicate a successful detection of the injection device 1 and/or the successful readout of respective device information or container information. Then, the external electronic device 100 may either provide an illustration 153 and/or a notification 154 to the user that a particular type of an injection device 1 has been registered or detected by the add-on device 30.

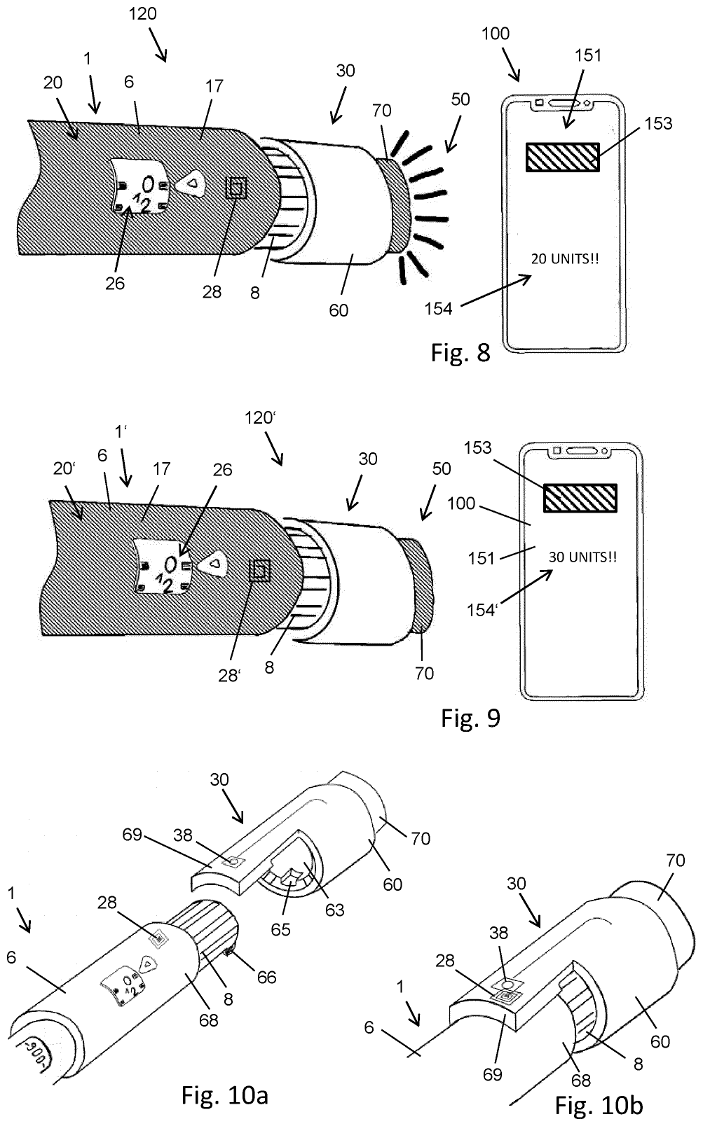

With some examples and as illustrated in FIGS. 8 and 9 after attaching the add-on device 30 to the injection device 1 the add-on device 30 is operable to quantitatively measure a size of a dose currently set or dispensed and to calculate a size of a respective dose on the basis of the sensor signals as well as on the basis of the device specific information or container specific information. Optionally, the movable part 70 or some other portion of the add-on device 30, e.g. a portion of the device body 60 may be provided with a separate indicator 50 operable to provide a user perceptible indication, e.g. of optical, audible or haptic type. This way a user can be informed that e.g. a dose setting procedure or dose dispensing or injection procedure is currently due, is currently in process or has just been completed. By way of the wireless communication between the add-on device 30 and the external electronic device 100 respective user assisting information can be provided in form of a notification 154 and/or an illustration 153 on the device display 151. With the example of FIG. 8 and when dialing the dose dial to a well-defined degree the notification may provide information about the size of the dose, e.g. 20 units.

Generally, the external electronic device 100 may permanently or regularly synchronize with the add-on device e.g. by local range communication interfaces as provided by the transceivers 39, 139. Insofar, any information gathered or read by the add-on device 30 can be instantly transmitted to and further process, e.g. visualized by the external electronic device 100, 100′.

With the further example as illustrated in FIG. 9 the add-on device 30 is assembled or attached to a different type of an injection device 1′. The injection device 1′ also comprises a machine-readable identification 28′ containing a different type of device information or container information. The injection device 1′ distinguishes from the injection device 1 as illustrated in FIG. 8 by the transmission ratio of its drive mechanism 20′ compared to the transmission ratio of the drive mechanism 20. For instance, the drive mechanism 20′ is dedicated or designed for a different type of a medicament. the transmission ratio may be characteristic for the degree of movement of the dose dial 8 relative to the degree of movement of the piston rod 19.

Since the electronic module 34 of the add-on device 30 is likewise configured to readout the machine-readable identification 28 when the add-on device 30 is assembled to the injection device 1′, respective device information and/or container information is obtained. The injection system 120′ as illustrated in FIG. 9 may be used in the same way as the injection device 1 or injection system 120 as illustrated in FIG. 8. Also here and for setting of a dose the dose dial 8 may be rotated by the same amount or degree as compared to the situation as described above in connection with FIG. 8.

Since the drive mechanism 20′ comprises a different mechanical transmission or transmission ratio compared to the drive mechanism 20 the equal movement of the dose dial 8 corresponds a different size of a dose, e.g. 30 units as illustrated on the device display 151. Here, the sensor signal as obtained from the sensor 48 may be equal with both situations as illustrated in FIGS. 8 and 9.

However, and since the add-on device 30 is attached to different injection devices 1, 1′ with drive mechanisms 20, 20′ comprising different transmission ratios between a dose setting motion of the dose dial 8 and a resulting feeding or advancing motion of the piston rod 19 the calculation or determination of the dose size has to be the recalibrated in accordance to at least one of the device specific information and the container specific information.

In FIGS. 10a and 10b there is illustrated a further implementation of an add-on device 30 for attaching to a proximal end of the body 6 of the injection device 1. Here, the device body 60 comprises a distally protruding extension 69, which overlaps with a machine-readable identification 28 as e.g. provided at or near the proximal end 68 of the body 6. The device body 60 comprises a somewhat tubular or sleeve sized and shaped to receive the dose dial 8 at the proximal end of the injection device 1. The extension 69 protruding distally from a distal end face of the tubular portion of the device body 60. By the device body 60 and hence its tubular portion is and remains fixed to the dose dial 8 located at a proximal offset from the body 6 of the injection device 1 that extension 69 comprises that a longitudinal dimension or extend that it reaches the device body 6, at least the proximal end of the device body 6. Insofar, a distal end of the extension 69 may overlap with the body 6. This way, and due to the laterally overlapping configuration of the extension 69 with the body 6 of the housing 10 perspective overlapping portions of the body 6 and the extension 69 can be provided with the near field transceiver is, i.e. with the electronic identifier 28 and/or with the near field transceiver 38.

Hence, the transceiver 38 of the electronic module 34 is located on or inside the longitudinal extension 69 so as to axially and/or radially overlap with the machine-readable identification 28 while a major or base part of the device body 60 is and remains attached to the dose dial 8 at least in such configurations, in which the injection device 1 is in a zero-dose configuration, e.g. when the dial extension 27 is in the distal end position.

Here, the machine-readable identification 28 may be provided on an inside surface or outside surface of the body 6. It may be provided in or on a label 17 adhered to the body 6.

As further illustrated in FIG. 10a, the receptacle 63 of the device body comprises an alignment feature 65 in form of a longitudinal recess or groove on the inside of the sidewall of the receptacle 63. Correspondingly, the proximal end of the dose dial 8 comprises a complementary shaped counter alignment feature 66, e.g. in form of a protrusion sized to fit into the recess or groove of the alignment feature 65. In this way, there can be provided a rotation inhibiting fastening for the add-on device 30 when attached to the housing 10 or dose dial 8 of the injection device 1.

When the alignment feature 65 is in mechanical engagement with the counter alignment feature 66 there is provided an anti-rotation lock for the mutual fastening of the add-on device 30 to the injection device 1. In this way there can be guaranteed, that the identification 28 and hence the electronic identifier 29 always correctly aligns with the associated wireless transceiver 38 of the add-on device 30. There is hence provided a unique and well-defined mounting position for mounting the add-on device 30 to the injection device 1. When reaching the mounting position or mounting configuration as predefined by alignment feature 65 engaging the counter alignment feature 66 it is also guaranteed, that the machine-readable identification 28 and the transceiver 38 are at a minimum distance to each other. This allows for a somewhat undisturbed signal transmission between the identification 28 or electronic identifier 29 and the transceiver 38.

Generally, the external electronic device 100 may permanently or regularly synchronize with the add-on device e.g. by local range communication interfaces as provided by the transceivers 39, 139. Insofar, any information gathered or read by the add-on device 30 can be instantly transmitted to and further processed, e.g. visualized, by the external electronic device 100, 100′.

With some examples the electronic module 34 may regularly check or regularly read the machine-readable identification 28, 88. A reading of at least one of the device information and the container information may be conducted on the basis of a predefined temporal schedule, such as once a day or once per hour. With some examples reading of the machine-readable identification by way of the electronic module 34 may be triggered each time the electronic module 34 is activated or when the electronic components of the electronic module 34 switch from an idle mode into an activated mode, e.g. when the electronic module 34 is subject to a wake up procedure.

The device information and/or container information may be at least temporally stored in the module memory 40 of the electronic module 34 during or in the course of mounting or attaching the add-on device 30 to the injection device 1. In the course of repeated use of the add-on device 30 the respective device information and/or container information may be repeatedly obtained from the machine-readable identification 28, 88. The actually obtained device information and/or container information may then be compared with the previously stored device information or container information. In case that the actually retrieved device information or container information does not match with the respective and previously stored device information or container information the add-on device 30 may be no longer correctly assembled to the injection device 1 or may have been reassembled to another injection device 1′.

In case of a detected mismatch between initially stored container information or initially stored device information with actually obtained device information or container information the electronic module 34 may activate its locking controller 86 so as to induce or to activate the locking mechanism 85 of the drive mechanism 20, thereby locking the operation of the drive mechanism 20.

With some examples it may be implemented that the locking mechanism 85 of the drive mechanism 20 is in a locking configuration per default and that the presence of the add-on device 30 at or on the injection device 1 is required to unlock the locking mechanism 85. In this way, a user or patient safety can be enhanced and misuse of the injection device 1 can be effectively prevented.

In the flowchart of FIG. 14 a method of monitoring operation of the injection device 1 as described herein is schematically illustrated. In a first step 200 the add-on device 30 is mounted on the injection device 1. With a second step 202 at least one of a device information and a container information is read or obtained from the machine-readable identification 28, 88 of the injection device by at least one of the reader 37 and the transceiver 38, 39 of the add-on device 30.

Thereafter, and in step 204 at least one of a position and a movement of a movable component 80, 81, 82, 83 of the drive mechanism 20 is quantitatively determined by the sensor thereby generating a respective sensor signal or a sequence of sensor signals.

In the following step 206 the sensor signal(s) obtained from the sensor 40 of the add-on device 30 are processed concurrently with the device information and/or container information in order to correctly calculate or obtain the size of a dose of the medicament currently set or injected.

### REFERENCE NUMBERS

1 injection device
2 distal direction
3 proximal direction
4 dose incrementing direction
5 dose decrementing direction
6 body
7 container part
8 dose dial
9 trigger
10 housing
11 connector
12 needle assembly
13 injection needle
14 inner needle cap
15 outer needle cap
16 protective cap
17 label
18 piston
19 piston rod
20 drive mechanism
21 medicament container
22 barrel
23 seal
24 medicament
25 outlet
26 window
27 dial extension
28 identification
29 electronic identifier
30 add-on device
34 electronic module
36 printed circuit board
37 reader
38 transceiver
39 transceiver
40 module memory
42 clock
44 module processor
46 power source
48 sensor
50 indicator
51 display
52 signal generator
53 display section
54 display section
60 device body
61 sidewall
62 flange portion
63 receptacle
64 fastening rib
65 alignment feature
66 counter alignment feature
70 movable part
80 dose setting element
81 drive member
82 volume indicator
83 encoding
85 locking mechanism
86 locking controller
88 identification
89 electronic identifier
90 electronic circuit
91 antenna
92 memory
93 integrated circuit
94 processor
95 substrate
96 power source
97 adhesive layer
100 electronic device
101 housing
103 wristband
110 hand
111 wrist
112 palm
114 thumb
116 finger
120 injection system
137 device reader
138 device transceiver
139 device transceiver
140 device memory
144 device processor
146 device power source
151 device display
152 device signal generator
153 illustration
154 notification

1.-18. (canceled)

19. An add-on device for attaching to an injection device comprising a housing, a drive mechanism for injecting of a dose of a medicament provided in a medicament container, the drive mechanism comprising at least one movable component, which is subject to a movement relative to the housing or relative to the medicament container during at least one of setting of the dose and injecting of the dose of the medicament, and a machine-readable identification containing at least one of a device information and a container information, the add-on device comprising:
a device body fastenable to the injection device,
a sensor operable to quantitatively determine at least one of a position and a movement of the movable component relative to at least one of the housing, the medicament container and the device body and operable to generate a respective sensor signal,
at least one of a reader and a transceiver being operable to read or to obtain the machine-readable identification of the injection device and
an electronic module comprising a module processor coupled to the sensor and to at least one of the reader and the transceiver, wherein the module processor is operable to determine or to calculate a size of the dose of the medicament, which is set or dispensed by the injection device, on the basis of the sensor signal and at least one of the device information and the container information contained in the machine-readable identification.

20. The add-on device according to claim 19, wherein the at least one of the reader and the transceiver comprises a wireless near-field transceiver operable to wirelessly communicate with the machine-readable identification of the injection device to obtain at least one of the device information and the container information.

21. The add-on device according to claim 19, wherein the at least one of the reader and the transceiver comprises a wireless local range transceiver, wherein the wireless local range transceiver is operable to wirelessly communicate with an external electronic device to receive the machine-readable identification of the injection device from or via the external electronic device.

22. The add-on device according to claim 19, wherein the module processor is operable to convert the sensor signal into dose size information by taking into account at least one of the device information and the container information.

23. The add-on device according to claim 19, wherein the module processor is operable to convert a first sensor signal into a first dose size information when provided with at least one of a first device information and a first container information and wherein the module processor is operable to convert the first sensor signal into a second dose size information when provided with at least one of a second device information and a second container information.

24. The add-on device according to claim 19, wherein the electronic module further comprises a module memory connected to the module processor and operable to store:
i) at least one of the device information and the container information as a stored information and/or
ii) a dosing history, the dosing history including a number of dose sizes dispensed or injected by the injection device and determined or calculated by the module processor.

25. The add-on device according to claim 24, wherein the module processor is operable to determine or to calculate a residual amount of the medicament contained in the medicament container on the basis of the dosing history and on the basis of at least one of the device information and the container information.

26. The add-on device according to claim 25, wherein the module processor is operable to discard or to interrupt determination or calculation of the residual amount of the medicament contained in the medicament container, if at least one of:
the device information obtained from the machine-readable identification does not match with the stored device information and
the container information obtained from the machine-readable identification does not match with the stored container information.

27. The add-on device according to claim 24, wherein the module processor is operable to compare at least one of the device information and the container information obtained from the machine-readable identification with the stored information.

28. The add-on device according to claim 27, wherein the module processor is operable to discard or to interrupt determination or calculation of the residual amount of the medicament contained in the medicament container if at least one of:
the device information obtained from the machine-readable identification does not match with the stored device information and
the container information obtained from the machine-readable identification does not match with the stored container information.

29. The add-on device according to claim 27, further comprising a signal generator, wherein the module processor is operable to activate the signal generator.

30. The add-on device according to claim 29, wherein the module processor is further operable to generate or to trigger an alert signal if at least one of:
the device information obtained from the machine-readable identification does not match with the stored device information and
the container information obtained from the machine-readable identification does not match with the stored container information.

31. The add-on device according to claim 19, wherein the module processor is operable to acquire and/or to request at least one of the device information and the container information before, during or after processing the sensor signals.

32. The add-on device according to claim 19, wherein the machine-readable identification comprises an electronic identifier and wherein the module processor is further operable to
generate use-related data being indicative of a status of use of the injection device;
to transmit the use-related data to the machine-readable identification; and
to store the use-related data in a memory of the electronic identifier.

33. An injection system comprising:
a first injection device comprising:
a first housing,
a first drive mechanism for injecting of a dose of a medicament provided in a first medicament container, the first drive mechanism comprising at least one movable component, which is subject to a movement relative to the first housing or relative to the first medicament container during at least one of setting of the dose and injecting of the dose of the medicament, and
a first machine-readable identification containing at least one of a first device information and a first container information, and
an add-on device, comprising:
a device body fastenable to the first injection device,
a sensor operable to quantitatively determine at least one of a position and a movement of the movable component relative to at least one of the housing, the first medicament container and the device body and to generate a respective sensor signal,
at least one of a reader and a transceiver being operable to read or to obtain the machine-readable identification of the injection device and
an electronic module comprising a module processor coupled to the sensor and coupled to at least one of the reader and the transceiver, wherein the module processor is operable to determine or to calculate a first size of the dose of the medicament, which is set or dispensed by the first injection device, on the basis of the sensor signal and on the basis of at least one of the first device information and the first container information contained in the machine-readable identification.

34. The injection system according to claim 33, further comprising a second injection device comprising:
a second housing,
a second drive mechanism for injecting of a dose of a medicament provided in a second medicament container, the second drive mechanism comprising at least one movable component, which is subject to a movement relative to the housing or relative to the second medicament container during at least one of setting of the dose and injecting of the dose of the medicament, and
a second machine-readable identification containing at least one of a second device information and a second container information,
wherein the module processor of the add-one device is operable to determine or to calculate a second size of the dose of the medicament, which is set or dispensed by the second injection device, on the basis of the sensor signal and on the basis of at least one of the second device information and the second container information contained in the second machine-readable identification.

35. The injection system according to claim 34, wherein at least one of the first and second machine-readable identifications comprises an electronic identifier comprising an electronic circuit comprising an antenna for wireless communication with at least one of the reader and the transceiver of the add-on device and comprising a memory to store at least one of the device information and the container information.

36. The injection system according to claim 35, wherein the memory is further configured to store use-related data being indicative of a status of use of the injection device.

37. A method of monitoring operation of an injection device configured for injecting of a dose of a medicament, the method comprising the steps of:
reading or obtaining at least one of a device information and a container information from a machine-readable identification of the injection device by at least one of a reader and a transceiver of an add-on device configured for attaching to the injection device,
quantitatively determining at least one of a position and a movement of a movable component of a drive mechanism of the injection device operable to inject a dose of the medicament provided in a medicament container and generating a respective sensor signal,
determining or calculating a size of the dose of the medicament, which is set or dispensed by the injection device on the basis of the sensor signal and on the basis of at least one of the device information and the container information.

38. A computer program comprising computer readable instructions, which when executed by a processor cause the processor to perform the steps of the method according to claim 37.
